(19) United States
(12) Patent Application Publication
(10) Pub. No.: US 2012/0190663 A1
(43) Pub. Date: Jul. 26, 2012

(54) USE OF FREE DNA AS AN EARLY PREDICTOR OF SEVERITY IN ACUTE PANCREATITIS

(75) Inventors: Olga Gornik, Zagreb (HR); Ivan Gornik, Zagreb (HR); Gordan Lauc, Zagreb (HR)

(73) Assignee: CEDARS-SINAI MEDICAL CENTER, Los Angeles, CA (US)

(21) Appl. No.: 13/389,154

(22) PCT Filed: Aug. 6, 2010
(86) PCT No.: PCT/IB10/01957

§ 371 Date: Apr. 16, 2012

Related U.S. Application Data
(60) Provisional application No. 61/231,937, filed on Aug. 6, 2009.
(60) Provisional application No. 61/309,319, filed on Mar. 1, 2010.

(51) Int. Cl. A61K 31/43; A61P 29/00; A61P 1/18; C12Q 1/68; A61P 31/00

(52) U.S. Cl. 514/192; 435/6.12

(57) ABSTRACT

This invention provides methods of determining the risk of AP development in an individual, as well as the diagnosis, prognosis, and treatment of acute pancreatitis (AP) in an individual, by determining the presence or absence of significantly high levels of free serum DNA in the subject relative to levels in a healthy individual. In other embodiments, the invention further provides methods of determining the risk of severe AP development, prognosis, diagnosis, and treatment of a severe form of acute pancreatitis based upon the presence of significantly high levels of free serum DNA in the subject relative to an individual who has and maintains a mild form of acute pancreatitis.

### FIELD OF THE INVENTION

The field of invention relates to a method of determining the risk of severe acute pancreatitis (AP) development in an individual, as well as diagnosing, prognosing, and treating severe AP in an individual by detecting the presence or absence of a significantly high level of free serum and/or free plasma DNA, where the presence of significantly high level of free serum and/or free plasma DNA in the individual is indicative of severe AP or an increased risk of severe AP development. The method can be used for differentiation of patients at high risk of severe AP.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Circulating (cell-free) DNA in serum or plasma has been investigated as a non-invasive diagnostic tool in a variety of clinical conditions. The investigations have mainly been focused on autoimmune diseases, cancer research and prenatal diagnostics of fetal diseases. However, elevated levels of circulating DNA have also been reported in acute medical conditions such as trauma, stroke, myocardial infarction and sepsis and explored as indicators of disease severity and predictors of mortality. New molecular methods, primarily quantitative real time PCR make quantisation of cell-free DNA a rapid and widely available potential diagnostic and prognostic tool. The origin of circulating DNA is not fully understood, but cell-death either by apoptosis or necrosis is believed to be the main source.

Acute pancreatitis (AP) is an acute inflammatory condition of the pancreas that may extend to local and distant extra-pancreatic tissues, and is most frequently caused by gallstone disease or excess alcohol ingestion. Gallstone pathogenesis is due to transient obstruction of the pancreatic duct orifice to the flow of pancreatic exocrine secretions; the pathogenesis resulting from excess alcohol consumption is less well understood. After recurrent episodes, most alcoholics go on to develop chronic pancreatitis. Other less common causes of AP include toxins, drugs, infections, trauma, vascular insults, anatomic abnormalities, and metabolic derangements. Hypertriglyceridemia and hypercalcemia are both implicated in AP, and serum triglyceride levels>1000 mg/dL have been found to precipitate an attack of AP, though the pathogenesis is not clearly understood. Hypercalcemia is thought to result from deposition of calcium in the pancreatic duct and calcium activation of trypsinogen. Idiopathic pancreatitis occurs in up to 20% of patients with AP, and the cause is not established by history, physical examination, routine laboratory tests, or imaging, although the majority of idiopathic cases of pancreatitis are thought to have a biliary source (Munsell et al., J Hosp Med 2010; 5(4):241-50).

AP is broadly classified as mild or severe, based on the classification system developed at the International Symposium on Acute Pancreatitis held in Atlanta (Bradley et al., Arch Surg 1993; 128:586-590). According to the Atlanta guidelines, AP is classified as severe if systemic and/or local complications are present; otherwise, AP is classified as mild. Mild or interstitial AP implies preservation of pancreatic blood supply. Necrosis suggests the disruption of pancreatic blood supply, with resulting ischemia. Severe AP may also imply the presence of organ failure, local complications such as pseudocyst or abscess, or pancreatic necrosis.

Organ failure occurs in half of patients with pancreatic necrosis. The most common organ failure in severe AP is respiratory failure, although other organs may fail, such as renal, hepatic, cardiovascular, digestive, neurologic, coagulation, endocrine, or immunologic systems. Failure of different organs can affect the prognosis of the disease. Mortality rates in severe AP patients with respiratory, renal, and hepatic failure are 43%, 63%, and 83%, respectively (Halonen et al. Crit Care Med 2002; 30:1274-1279). As many as 37%-76% of AP patients who will develop organ failure may have it already at admission or develop it during the first 24 hours. Therefore, the ability to detect AP or the risk of AP during the initial 24 hours of admission is critical in preventing further damage to the organs that may have been initiated by AP development. Delayed admission to the intensive care unit results in a 4-fold excess in mortality, and early identification of patients who develop a severe AP with organ failure is essential for improved prognosis by earlier intervention with appropriate treatment regimen. At present, no specific medical therapy for AP exists (Kylanpaa et al. World J Gastroenterol 2010; 16(23):2867-72), but supportive care with organ replacement treatments in an ICU is essential in severe AP.

The diagnosis of AP is often based on elevated serum levels of pancreatic enzymes such as amylase and lipase. The elevation of these enzymes, however, is not pathognomonic for the presence of disease. These enzymes may not always be significantly elevated during times of acute inflammation, and elevation of the enzymes can come from nonpancreatic origins. Isoamylase levels can be used to distinguish among pancreatic, salivary, and macroamylasemia though this is not often used if pancreatitis is suspected clinically. Similarly, serum isolipase can be measured, though this is not readily available.

AP, especially in cases of severe disease, is characterised by necrosis of pancreatic tissue, accompanied by intensive inflammation and further tissue damage, in part mediated by microvasculature occlusions. AP is usually a self-limiting, short-lasting, mild disease, but in some 20% of cases the disease takes a severe course with systemic inflammatory response, local and systemic complications and high mortality rates despite treatment. Early identification of severe AP remains a serious problem in clinical practice. Clinical judgment alone has good specificity but low sensitivity, since it misses many severe cases. Development of a method for early recognition of AP, especially severe cases of AP, is a major issue, since early treatment may reduce morbidity and mortality.

Ranson and colleagues developed the first scoring system that was used for predicting severity of AP more than 30 years ago (Ranson et al., Surg Gynecol Obstet 1974; 139:69-81). Since then, other multifactorial systems, like the Glasgow (Imrie) score, have been developed specifically for that purpose. Some non-specific scores, like acute physiology and chronic health evaluation (APACHE) II score, have also been used as predictors of disease course as well as single biochemical markers. These mainly include markers of inflammation such as C-reactive protein (CRP), interleukin (IL) 6, IL-8, TNF-alpha, and HLA-DR expression on monocytes. Of those, CRP measurement is the most widely accepted single test severity marker, but it usually rises only after the second day of disease and is not specific. Abdominal CT, performed during the first days of disease also has predictive significance in AP. Other parameters and scoring systems have also been investigated, usually with small additional predictive value or none at all.

Thus, there is a need in the art for new molecular methods directed at diagnosing, prognosing and/or treating acute pancreatitis, specifically by determining the extent of cell-free DNA.

### SUMMARY OF THE INVENTION

In an embodiment, the invention includes a method of determining the likelihood of acute pancreatitis (AP) development in an individual, comprising: obtaining a sample from the individual; assaying the sample to detect the presence or absence of a significantly high level of cell-free DNA in the sample; determining a high risk of AP development in the individual based upon the presence of a significantly high level of cell-free DNA in the sample or diagnosing a low risk of AP development in the individual based upon the absence of a significantly high level of serum DNA in the sample. The sample may be a serum sample. The sample may be a plasma sample. The use of serum and the use of plasma are separate and distinct embodiments of the invention. A serum sample can be obtained using different methods from the methods used to obtain a plasma sample. The significantly high level of cell-free DNA may be a level above 0.050 ng/μl, a level above 0.100 ng/μl, a level above 0.150 ng/μl, or a level of at least 0.271 ng/μl, or a level of at least 0.304 ng/μl, or a level of at least 0.363 ng/μl.

The concentration of serum DNA used as an indicator of the severity of AP can be different from the concentration of plasma DNA used as an indicator of the severity of AP. The AP may be a severe form of AP. The AP may be one of several episodes of AP in a patient with chronic pancreatitis. The method may further include detecting gallstones in the individual prior to obtaining the sample. The method may further include detecting one or more stimulants in the individual prior to obtaining the sample. The stimulants may be selected from toxins, drugs, infections, trauma, vascular insults, anatomic abnormalities, metabolic derangements and combinations thereof. The method may further include detecting hypertriglyceridemia and/or hypercalcemia in the individual prior to obtaining the sample. The method may further include determining a likelihood of developing a related complication selected from the group consisting of pancreatic necrosis, tissue abscess, glandular damage, and diabetes mellitus. The sample may be obtained from the individual on the day that the individual is admitted to a hospital.

In another embodiment, the invention includes a method of determining the prognosis of a subject with pancreatitis, comprising: obtaining a sample from the subject; assaying the sample to detect the presence or absence of a significantly high level of cell-free DNA in the sample relative to either a healthy individual or an individual who has and maintains a mild form of acute pancreatitis; and prognosing a form of pancreatitis that will progress to severe AP based upon the presence of the significantly high level of cell-free DNA in the subject relative to either the healthy individual or the individual who has and maintains a mild form of acute pancreatitis. The sample may be a serum sample. The sample may be a plasma sample. The significantly high level of cell-free DNA may be a level above 0.050 ng/μl, a level above 0.100 ng/μl, a level above 0.150 ng/μl, or a level of at least 0.271 ng/μl, or a level of at least 0.304 ng/μl, or a level of at least 0.363 ng/μl. The sample may be obtained from the individual on the day that the individual is admitted to a hospital.

In another embodiment, the invention includes a method of treating acute pancreatitis in a subject, comprising: obtaining a sample from the subject; assaying the sample to detect the presence or absence of a significantly high level of cell-free DNA in the sample; determining a high risk of AP development in the individual based upon the presence of a significantly high level of cell-free DNA in the sample or diagnosing a low risk of AP development in the individual based upon the absence of a significantly high level of serum DNA in the sample; and treating the subject to either prevent or lessen the likelihood of developing or mitigate the effects of AP. The sample may be a serum sample. The sample may be a plasma sample. The significantly high level of cell-free DNA may be a level above 0.050 ng/μl, a level above 0.100 ng/μl, a level above 0.150 ng/μl, a level of at least 0.271 ng/μl, or a level of at least 0.304 ng/μl, or a level of at least 0.363 ng/μl. The sample may be obtained from the individual on the day that the individual is admitted to a hospital.

In another embodiment, the invention includes a kit for determining the likelihood of acute pancreatitis (AP) development in an individual, comprising: one or more components to obtain a blood sample from the individual; one or more components to isolate plasma or serum from the blood sample; one or more components to determine a level of plasma DNA or serum DNA; and instructions with tangible expression describing a technique, comprising obtaining the blood sample from the individual, isolating plasma and/or serum from the blood sample, determining a significantly high level of plasma DNA and/or serum DNA in the blood sample, and determining a high risk of AP development in the individual based upon the presence of a significantly high level of plasma DNA or serum DNA in the blood sample.

In another embodiment, the invention includes a kit for treating acute pancreatitis (AP) in an individual, comprising: one or more components to obtain a blood sample from the individual; one or more components to isolate plasma or serum from the blood sample; one or more components to determine a level of plasma DNA or serum DNA; instructions with tangible expression describing a technique, comprising obtaining the blood sample from the individual, isolating plasma or serum from the blood sample, determining a significantly high level of plasma DNA or serum DNA in the blood sample, and determining a high risk of AP development in the individual based upon the presence of a significantly high level of plasma DNA or serum DNA in the blood sample; and one or more components for treating AP.

In another embodiment, the invention includes a method of assessing severity of episodes of AP in patients with chronic pancreatitis, who have recurrent episodes of AP. The method can include obtaining a sample from a subject; assaying the sample to detect the presence or absence of a significantly high level of cell-free DNA in the sample relative to either a healthy individual or an individual with chronic pancreatitis; and determining the severity of pancreatitis of the AP episode based upon the presence of the significantly high level of cell-free DNA in the subject relative to either the healthy individual or the individual with chronic pancreatitis. The sample may be a serum sample. The sample may be a plasma sample. The significantly high level of cell-free DNA may be a level above 0.050 ng/μl, a level above 0.100 ng/μl, a level above 0.150 ng/μl, or a level of at least 0.271 ng/μl, or a level of at least 0.304 ng/μl, or a level of at least 0.363 ng/μl. The sample may be obtained from the individual on the day that the individual is admitted to a hospital.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment herein, comparison of free serum DNA concentrations in control (N=18) patients with mild (N=21) and severe (N=9) acute pancreatitis measured on admission, and on the first, fourth and seventh days following admission. Data are presented with box and whisker plots (median, quartiles, range), with dots representing individual values: severe acute pancreatitis—black lines and black dots; mild acute pancreatitis—grey lines with grey dots, controls—black lines with white dots.

FIG. 2 depicts, in accordance with an embodiment herein, a receiver operating characteristic (ROC) curve of the free serum DNA on the first day after admission in distinguishing patients with mild and severe acute pancreatitis.

FIG. 3 depicts, in accordance with an embodiment herein, data comparison graphs for concentrations of cell free DNA in A) plasmas and B) sera of patients who developed mild and severe AP. Data are presented with box and whisker plots (median, quartiles, range).

FIG. 4 depicts, in accordance with an embodiment herein, prediction of severe acute pancreatitis using plasma (full line) and serum (dashed line) free DNA on first day after admission.

FIG. 5 depicts, in accordance with an embodiment herein, comparison of free DNA from plasma and serum with methods currently used for severity assessment/prediction. AUC—area under ROC curve; PLASMA—free DNA in plasma; SERUM—free DNA in serum; CRP—C-reactive protein; APACHE II—acute physiology and chronic health evaluation II score; Ranson—Ranson's score.

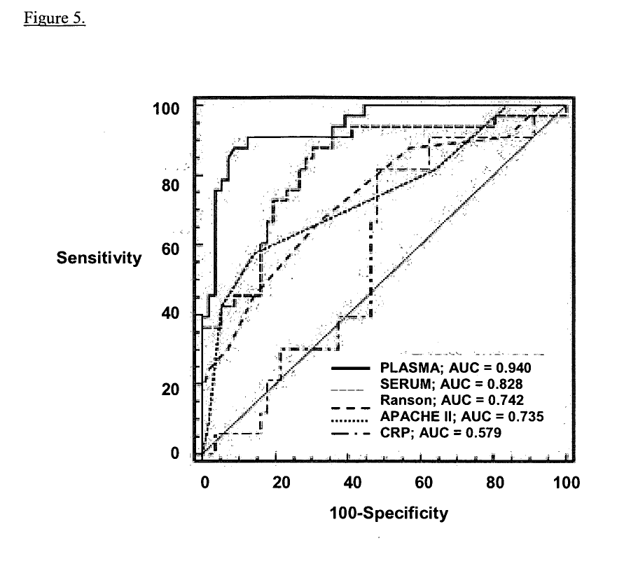

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

Circulating (cell-free) DNA can be found in healthy persons and persons with nonmalignant diseases, including systemic lupus erythematosus, rheumatoid arthritis, pulmonary embolism, or myocardial infarction and various malignancies. Trauma and therapeutic procedures have also been shown to lead to the release of free DNA into the circulation, although the actual origin remains unknown. The reported values for reference concentrations of DNA in healthy controls range from barely detectable concentrations to a few micrograms per liter. In healthy persons, it can be presumed that circulating DNA originates from lymphocytes or other nucleated cells, but its origin in malignancies is still unknown (Pathak et al. 2006; 52:1833-42).

As disclosed herein, the inventors tested free DNA levels in serum and plasma as prognostic markers for severity of acute pancreatitis. Plasma refers to the liquid portion of normal unclotted blood containing the red and white cells and platelets, as well as dissolved proteins, glucose, and clotting factors. Serum is the clear liquid that can be separated from clotted blood, and is devoid of clotting factors. Plasma and serum are obtained from blood samples of patients, which are removed from the circulatory system via venipuncture. To obtain a plasma sample, the blood sample is withdrawn in the presence of an anticoagulant and centrifuged to remove cellular elements. To obtain a serum sample, the blood sample is obtained in the absence of anticoagulant, and after the blood clots, the sample can be centrifuged to remove the fibrin clot and cellular elements. Blood can also be withdrawn through a resin-containing device which depletes the blood of calcium precluding coagulation. Other methods of preventing or slowing coagulation include the addition of corn trypsin inhibitor, common protease inhibitor mixtures, anticoagulants such as EDTA (ethylenediamine tetraacetic acid), sodium citrate, and heparin. Plasma and serum can also be obtained by taking a blood sample, separating plasma by centrifugation immediately after blood draw, and separating the serum after allowing the blood to clot. Sera and plasma samples can be stored at −80° C. until further analysis.

The method used to obtain serum can be different from the method used to obtain plasma. The use of serum as an indicator of the severity of AP, in diagnosis, prognosis, or treatment of AP can be different from the use of plasma as an indicator of severity of AP in diagnosis, prognosis, or treatment of AP. The levels of free serum DNA used as an indicator can be different from the levels of free plasma DNA used as an indicator. The use of serum and the use of plasma are separate and distinct embodiments of the invention.

Several methods exist for the quantification of serum and plasma DNA. Colorimetric or fluorometric assays are performed by adding reagents such as diphenylamine to the patient's plasma or serum, which produces a color change, the degree of which correlates with the DNA concentration. Other assays include hemagglutinin inhibition, complement fixation, and diffusion in agarose. With the more sensitive RNA-DNA hybridization, RIA, and counterimmunoelectrophoresis assays, nanogram amounts of circulating DNA can be quantified with high accuracy. With real-time PCR and PicoGreen double-stranded DNA quantification assays, picogram amounts of free DNA can be quantified. Those skilled in the art will readily appreciate various methods to obtain and test free serum and plasma DNA; the methods suggested are merely for purposes of example.

Serum or plasma DNA can be used alone, or serum and plasma DNA can both be used together as markers of the severity of AP in diagnosis, prognosis, or treatment of AP.

Results show that cell-free DNA measured in both plasma and serum of patients with acute pancreatitis is significantly higher in patients who developed severe acute pancreatitis than in those with mild disease. As further described herein, both plasma and serum levels of free DNA predict severity of AP more effectively than other common markers and scoring systems. The prediction of severe pancreatitis using free plasma DNA had a sensitivity of 90.9% and specificity of 88.7% when the cut-off value of >0.304 ng/μl was used in the inventors' experiments. The prediction using serum DNA had a sensitivity of 87.9% and specificity of 67.6% with a cut-off value of >0.363 ng/μl.

In some embodiments, free DNA was measured in sera from 30 patients with AP at admission, and on the first, fourth and seventh days following admission. On the first day following admission, patients who would develop severe AP had significantly higher serum DNA levels than those with mild disease (median 0.271 ng/μl vs. 0.059 ng/μl respectively; P<0.001). This parameter showed very good characteristics as a severity predictor (area under ROC curve 0.97). Free serum DNA was in correlation with the extent of pancreatic necrosis. Thus, in some embodiments, free DNA correlates with the extent of pancreatic necrosis and is thus an early marker of severe acute pancreatitis.

The present invention also pertains to the field of predictive medicine in which diagnostic assays, prognostic assays, and continuous monitoring are used for predictive purposes to thereby treat an individual. The invention provides a method of determining whether a subject is at risk for AP, and whether a subject is at risk for severe AP.

The method can include one or more of the following:

detecting, in the plasma obtained from the blood sample of the subject, the levels of free DNA, and/or detecting, in the serum obtained from the blood sample of the subject, the levels of free DNA;

comparing the levels of free DNA with the levels found in healthy subjects; and

monitoring the levels of free DNA in serum and/or plasma through the treatment of a subject to determine the effectiveness of the treatment regimen.

In other embodiments, the level of free DNA correlates with the extent of pancreatic necrosis and related complications such as tissue abscess, glandular damage, and diabetes mellitus. Acute pancreatitis inflammation can lead to pancreatic cell death, or pancreatic necrosis. This necrotized tissue can become infected, resulting in infected necrosis. Pancreatic necrosis may lead to the development of pancreatic pseudocysts or tissue abscess, common complications associated with pancreatitis. Pancreatic insults such as alcohol, gallstone disease, and smoking cause repeated pancreatic injury, and can exacerbate the extent of disease and promote development of permanent glandular damage. The pancreas is a key component in the regulation of blood sugar levels, and the development of diabetes mellitus is another major complication resulting from chronic pancreatitis or severe acute necrotizing pancreatitis. Pancreatitis directly causes diabetes as a result of inflammation-induced damage to islet cells, the insulin-producing cells of the pancreas (Banks et al. Gastroenterol Hepatol 2010; 6(2_Suppl): 1-16).

In some embodiments, the present invention provides a method of determining the likelihood of AP development in an individual by determining the presence or absence of a significantly high level of free serum DNA, where the presence of the significantly high level of free serum DNA in the individual is indicative of a high risk of AP development and the absence of such a level of free serum DNA is indicative of a reduced risk of AP development. In some embodiments, the significantly high level of free serum DNA is above 0.050 ng/μl. In other embodiments, the significantly high level of free serum DNA is above 0.100 ng/μl. In other embodiments, the significantly high level of free serum DNA is above 0.150 ng/μl. In other embodiments, the significantly high level of free serum DNA is above approximately 0.271 ng/μl.

In some embodiments, the present invention provides a method of prognosing a severe form of AP in an individual by determining the presence or absence of a significantly high level of free serum DNA, where the presence of significantly high level of free serum DNA in the individual is indicative of a prognosis of the severe form of acute pancreatitis. In other embodiments, significantly high level of free serum DNA is above 0.050 ng/μl. In other embodiments, significantly high level of free serum DNA is above 0.100 ng/μl. In other embodiments, significantly high level of free serum DNA is above 0.150 ng/μl. In preferred embodiments, significantly high level of free serum DNA is approximately 0.271 ng/μl. In other embodiments, the level of free serum DNA correlates with the extent of pancreatic necrosis.

In some embodiments, the present invention provides a method of determining the likelihood of AP development in an individual by determining the presence or absence of a significantly high level of free plasma DNA, where the presence of the significantly high level of free plasma DNA in the individual is indicative of a high risk of AP development and the absence of such a level of free plasma DNA is indicative of a reduced risk of AP development. In some embodiments, the significantly high level of free plasma DNA is above 0.050 ng/μl. In other embodiments, the significantly high level of free plasma DNA is above 0.100 ng/μl. In other embodiments, the significantly high level of free plasma DNA is above 0.150 ng/μl. In other embodiments, the significantly high level of free plasma DNA is above approximately 0.271 ng/μl.

In some embodiments, the present invention provides a method of prognosing a severe form of AP in an individual by determining the presence or absence of a significantly high level of free plasma DNA, where the presence of significantly high level of free plasma DNA in the individual is indicative of a prognosis of the severe form of acute pancreatitis. In other embodiments, significantly high level of free plasma DNA is above 0.050 ng/μl. In other embodiments, significantly high level of free plasma DNA is above 0.100 ng/μl. In other embodiments, significantly high level of free plasma DNA is above 0.150 ng/μl. In preferred embodiments, significantly high level of free plasma DNA is approximately 0.271 ng/μl. In other embodiments, the level of free plasma DNA correlates with the extent of pancreatic necrosis.

In some embodiments, the present invention provides detecting a change in the levels of free serum DNA and/or free plasma DNA, in the diagnosis, prognosis, and treatment of AP. Further embodiments involve the monitoring of therapy and treatment course using absolute values of free serum DNA and/or free plasma DNA.

In some embodiments, the present invention provides a method of treating AP in an individual in need thereof by determining the presence or absence of a significantly high level of serum DNA, and/or determining the presence or absence of a significantly high level of free plasma DNA, and treating the individual.

Embodiments of the present invention provide several advantages as a diagnostic tool, prognostic tool, and treatment method. The levels of free serum DNA and plasma DNA can be used alone or in combination as a predictor of AP development, and as a predictor of a severe form of AP development. Patient blood can be assessed at various time points for continuous monitoring, such as day 1 of hospital admission, day 2, day 4, day 7, or later. In some embodiments the invention can be in the form of a kit for measuring the risk of AP and severe AP, and providing appropriate treatment. The method can be used in determining the severity of episodes of AP in patients with chronic pancreatitis.

The primary treatment for AP is pancreatic rest, analgesia and restoration of a normal balance of fluid and electrolytes. Primary goals of treatment are close supportive care and prevention of pancreatic necrosis, infection, and organ failure. Supportive care includes pain control, fluid resuscitation, and nutritional support. Because infection of pancreatic necrosis dramatically increases the mortality rate of AP, a major goal in disease management is the prevention of infection. For early initiation of broad-spectrum antibiotics in preventing pancreatic infection, antibiotics with good pancreatic tissue penetration can be used, such as imipenem, cefuroxime, or ciprofloxacin (Stevens et al. Cleveland Clinic Dis Management Proj 2009). Treatment also includes close monitoring of vital signs such as pulse, blood pressure, rate of breathing, and urine production, and repeated blood draws to monitor the hematocrit, glucose levels, electrolyte levels, white blood cell count, and enzyme levels. When AP results from gallstones, treatment depends on the severity. For mild AP, removal of the gallbladder can usually be delayed until symptoms subside. Severe pancreatitis caused by gallstones can be treated with endoscopic retrograde cholangiopancreatography (ERCP).

The present invention is also directed to a kit for diagnosing or prognosing AP and/or the risk of AP development in a subject by obtaining blood samples upon admission and at various time points, obtaining the serum and plasma components of the blood samples (for example, by centrifugation and separation after coagulation), quantifying the levels of free serum DNA and/or free plasma DNA, comparing them to amounts of DNA found in healthy subjects (for example, by performing receiver operating curve (ROC) analysis for free DNA levels to obtain positive and negative likelihood ratios), and diagnosing, prognosing, or determining the likelihood of the subject developing severe AP. The kit is an assemblage of materials or components, including at least one of the inventive compositions. The kit can contain necessary instruments for venipuncture and blood sample storage, anticoagulants such as EDTA (ethylenediamine tetraacetic acid), sodium citrate, heparin, and the like. The kit can further contain components necessary for DNA isolation and quantification, such as proteinase K, buffers, S blocks, elution tubes, primers for DNA amplification, probes labelled with a dye, components for an internal PCR control assay, and the like. The kit can be used diagnostically to monitor levels of free serum DNA and/or free plasma DNA as part of a clinical testing procedure, e.g., to, for example, determine the efficacy of a given treatment regimen.

The present invention is also directed to a kit for the treatment of AP, which includes each component included in the diagnostic/prognostic kit described in the previous paragraph, and components that can be used for treatment, such as analgesics, antibiotics, nasogastric tube for fluid resuscitation and nutritional support, and the like.

The exact nature of the components configured in the inventive kit depends on its intended purpose. In some embodiments, the kit is configured particularly for the purpose of treating mammalian subjects. In other embodiments, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to determine the presence of serum DNA and/or free plasma DNA. Optionally, the kit can also contain other useful components, such as, but not limited to, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in detecting the presence of polynucleotides such that might be found in serum or plasma. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing antibodies, markers, etc. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Free Serum DNA Study

The inventors investigated free serum DNA obtained from AP patients to determine its correlation with the extent of pancreatic necrosis and as an early marker of severity. Free serum DNA was measured in sera from 30 patients with acute pancreatitis at admission, on the first, fourth and seventh day following admission. On the first day following admission, patients who would subsequently develop severe pancreatitis had significantly higher serum DNA levels than those with mild disease (median 0.271 ng/μl vs. 0.059 ng/μl respectively; P<0.001). This parameter showed very good characteristics as a severity predictor (area under ROC curve 0.97). Free serum DNA was also in correlation with the extent of pancreatic necrosis. Thus, free serum DNA was determined to correlate with the extent of pancreatic necrosis and is an early marker of severe acute pancreatitis.

### Example 2

### Comparative Serum Plasma DNA Study

The inventors investigated free serum DNA and free plasma DNA obtained from patients with AP to determine the correlation of each with the extent of pancreatic necrosis and as an early marker of severity of AP. In previous studies on a small (30 AP patients, 18 controls), carefully selected population (Gomik, et al., Clinical biochemistry 2009; 42:38-43), the inventors obtained a very high “post-hoc” calculated power of the study (100% power; beta error 0% alpha error 1%). Therefore, the inventors widened the inclusion criteria, and used a smaller but still significant difference, on day 4 from the previous study, as the reference for the sample size calculations. The sample size with alpha error of 1% and beta error of 5% (power of 95%) calculates to 51, which, after correcting for unequal distribution of mild (cca 80%) and severe (cca 20%) cases of acute pancreatitis, rises to 70.

### Example 3

### Patients and Control Group

A study on the serum DNA and a comparative study on plasma and serum DNA were conducted at the Department of Medicine, University Hospital Centre Zagreb. Adult patients (≧18 years) diagnosed with acute pancreatitis, regardless of etiology, were considered. Inclusion criteria were: clinical presentation consistent with acute pancreatitis (abdominal pain, nausea, vomiting, etc.); more than threefold elevation of serum amylase activity; and more than fivefold elevation of urine amylase activity. Patients with terminal illnesses and patients in whom pancreatitis was not a primary admission diagnosis or in whom pancreatitis was a manifestation of other acute or chronic conditions (e.g., pancreatitis in trauma or pancreatitis during chemotherapy) were excluded. Pregnant patients were also excluded. For the study on serum DNA, patients with other possible causes of elevated amylase activity (e.g., renal impairment, hyperlipidemia) were excluded. Elevation (more than threefold) of serum lipase activity was used to confirm the diagnosis.

Persons included in the control group were matched for sex and age, generally healthy, without any chronic or acute illness. Normal serum amylase and CRP values, and normal erythrocyte sedimentation rate (ESR) were additional criteria.

TABLE 1
Characteristics of patients and control subjects in serum study

| Parameter | Controls | | All patients | | Mild AP | | Severe AP | | mild vs. severe |
|---|---|---|---|---|---|---|---|---|---|
| N (%) | 18 | | 30 | | 21 | (70%) | 9 | (30%) | |
| Age (years) | 56.5 | (26-82) | 51 | (20-86) | 49 | (20-82) | 69 | (24-80) | P = 0.288 |
| Sex (female) | 9 | (50%) | 16 | (54.4%) | 12 | (57.1%) | 5 | (55.6) | P = 0.748 |
| Etiology | | | | | | | | | |
| alcohol | | | 15 | (50%) | 10 | (47.6%) | 5 | (55.6%) | P = 0.685 |
| gallstones | | | 13 | (43.3%) | 10 | (47.6%) | 3 | (33.3%) | |
| other | | | 2 | (6.7%) | 1 | (4.8%) | 1 | (11.1%) | |
| Survivors LOS (days) | | | 10 | (6-24) | 9 | (6-15) | 20 | (14-34) | P < 0.001 |
| Hospital mortality | | | 2 | (6.7%) | 0 | (0%) | 2 | (22.2%) | P = 0.151 |

TABLE 2
Characteristics of patients in comparative sera-plasma DNA study

| | | | | | | | Mild vs. |
|---|---|---|---|---|---|---|---|
| Parameter | All patients | | Mild AP | | Severe AP | | severe |
| N (%) | 104 | | 71 | (68%) | 33 | (32%) | |
| Age (years) | 54 | (26-74) | 54 | (26-74) | 55 | (27-74) | P = 0.511 |
| Sex (female) | 43 | (41%) | 29 | (41%) | 14 | (42%) | P = 0.951 |
| Etiology | | | | | | | |
| gallstones | 51 | (49%) | 34 | (48%) | 17 | (52%) | P = 0.829 |
| alcohol | 45 | (43%) | 33 | (47%) | 12 | (36%) | |
| other | 8 | (8%) | 4 | (6%) | 4 | (12%) | |
| Days from the | | | | | | | |
| onset of | | | | | | | |
| symptoms | | | | | | | |
| 1 | 53 | (51%) | 41 | (57.7%) | 12 | (36.4%) | P = 0.023 |
| 2 | 39 | (37.5%) | 26 | (36.6%) | 13 | (39.4%) | |
| 3 | 7 | (6.7%) | 3 | (4.2%) | 4 | (12.1%) | |
| 4 | 5 | (4.8%) | 4 | (5.6%) | 4 | (12.1%) | |

### Example 4

### Blood Sampling

For the serum DNA study, blood samples were taken from patients with acute pancreatitis on four occasions: at the moment of inclusion (Day 0=day of admission to hospital), on the first day following admission (Day 1), and on the fourth and seventh days of hospitalization. Immediately after blood draw, sera were separated by centrifugation and stored until analysis at −80° C.

For the comparative sera-plasma DNA study, blood samples were taken from patients with acute pancreatitis on the first day following admission. Plasma was separated by centrifugation immediately after blood draw; sera were separated after allowing the blood to clot for 15 minutes. Sera and plasma samples were stored until analysis at −80° C.

### Example 5

### Disease Course and Outcome

Age, sex and etiology of pancreatitis were noted for all the patients; Ranson's score was determined in the first two hospital days (Ranson et al., Surg Gynecol Obstet 1974; 139:69-81). CRP was measured on the first two days to be evaluated as a predictor of severity and later during the course of disease at the discretion of attending physicians. Commercial Olympus quantitative immunoturbodimetric latex test on Olympus AU400 analyser was used.

For the serum study, APACHE II score was calculated at inclusion on the days of blood sampling (days 1, 4 and 7) and later in the case of disease progression. For the comparative serum-plasma study, APACHE II score was calculated during the first 24 hours and later in the case of disease progression. The highest CRP value and APACHE II score during the hospitalization were noted as measures of disease severity.

Severe pancreatitis, organ failure and presence of local complications were defined according to the Atlanta 1992 criteria (Bradley, Ann Chir 1993; 47:537-41). Total hospital length of stay (LOS) and hospital mortality were used as outcome measures. Imaging methods were performed at the discretion of the attending physicians, who generally followed hospital guidelines: abdominal ultrasound was performed in all patients within the first day after admission and later if required. Abdominal CT was performed if there was clinical or ultrasonographic suspicion of pancreatic necrosis and/or hemorrhage or if clinical condition required. Images were reported according to the Balthazar scoring system (Balthazar et al. Radiology 1994; 193:297-306).

### Example 6

### Free Serum DNA Assay

DNA isolation: DNA was extracted from 100 μl of serum using a DNeasy Blood and Tissue Kit (QIAGEN GmbH, Germany) according to the manufacturer's protocol.

DNA quantification: Real time PCR was performed using a Quantifier Human DNA Quantification kit (Applied Biosystems, USA). This quantification assay combines two 5′-nuclease assays: a target specific assay and an internal PCR control (IPC) assay. The target specific assay consists of two primers for amplifying human DNA and one TaqMan MGB probe labeled with FAM dye for detection of the amplified sequence. The IPC assay consists of IPC template DNA (a unique synthetic sequence not found in nature), two primers for amplifying IPC template DNA and one TaqMan MGB probe labeled with VIC dye for detection of the amplified IPC DNA. Both target and IPC detectors are designed to amplify in parallel in every reaction. A coding region of the human telomerase reverse transcriptase gene (hTERT locus located on chromosome 5) was used to quantify total DNA.

Reaction conditions were as follows: 12.5 μl of Quantifier PCR Reaction Mix (containing NTP's, buffer, AmpliTaq Gold DNA polymerase and ROX Passive reference Standard), 10.5 μl of Quantifier Human Primer Mix (containing target specific primers, FAM labeled probe, IPC template, IPC primers and VIC labeled probe) and 2 μl of DNA extract in a final reaction volume of 25 μl. Thermal cycler conditions were: denaturation at 95° C. for 10 minutes followed by 40 cycles at 95° C. for 15 s and 60° C. for 60 s.

Amplification data were collected and analyzed with an ABI Prism 7000 Sequence Detection System (SDS) instrument (Applied Biosystems, USA). The cycle threshold value (Ct) was measured in all cases. Each sample was analyzed in duplicate, and multiple negative reaction blanks were included in every analysis for both sample extraction and amplification stages. Calibration curves (duplicate samples) were analyzed on the same reaction plate for each run. Quantification standard dilutions were made by serially diluting the 200 ng/μl stock solution from the kit (human male genomic DNA) to the following concentrations: 50, 16.7, 5.56, 1.85, 0.62, 0.21, 0.068, 0.023 and 0.0076 ng/μl.

### Example 7

### Statistical Analyses

MecCalc v. 7.2.1.0 statistical software was used for all statistical analyses. Non-parametric tests were used regardless of the distribution type for any measured parameter. Continuous variables are presented as medians with inter-quartile ranges, categorical as absolute and relative frequencies. Wilcoxon's test (paired and unpaired, as appropriate) was used for group comparisons of continuous variables; chi-squared test for categorical variables. Spearman's rank correlation was used for correlation analyses. Significance was set at α=0.05. Receiver operating characteristic (ROC) curves were constructed for evaluation of prognostic values of different variables. Areas under ROC curve (with 95% CI), sensitivity and specificity, positive and negative predictive values, positive and negative likelihood ratios were calculated for each examined parameter. The optimal cut-off point with best relationship between sensitivity and specificity was determined from the ROC curve.

DNA isolation and quantification from sera and plasma samples were performed by an experienced researcher in a research laboratory. At the moment of analysis and reading of the results the analyst was not aware of clinical conditions or any other information about the patients. These studies were performed in conformance to the ethical guidelines of the 1975 Declaration of Helsinki and approved by the ethics committees of the Clinical Hospital Centre Zagreb and the University of Osijek School of Medicine.

### Example 8

### Overall Results of the Serum Study

During the collection period of two months, 46 patients fulfilling the inclusion criteria were asked to participate in the study, 30 agreed and were included. Basic characteristics of patients and control group are summarized in Table 1. Median age was 50.5 (20-86) years, and 14 patients (46.6%) were male. Eighteen people (9 male) were included in the control group which did not differ from the patients group in age (P=0.288) or sex distribution (P=0.748). Nine patients (30%) met the criteria for severe acute pancreatitis, of which two (22.2%) had died. There were no significant differences in age, sex distribution or etiology between patients with mild and severe disease. Patients with severe disease, as expected, had longer hospitalization than those with mild disease (median 18 vs. 9 days, respectively).

### Example 9

### Overall Results of Comparative Serum Plasma Study

During the collection period of eight months, 125 patients fulfilling the inclusion criteria were asked to participate in the study, 104 agreed and were included. Basic characteristics of patients are summarized in Table 2.

During hospital stay, 71 (68%) of these patients developed mild pancreatitis, while 33 (32%) met the criteria for severe acute pancreatitis. Six patients died (18% of patients with severe disease).

There were no significant differences in age or sex distribution, or etiology between patients with mild and severe disease (Table 1). The majority of patients (89%) reported to the hospital within the first two days of feeling ill. Patients who came to the hospital later in the course of disease had a higher chance of having severe disease.

Thirteen (39%) patients with severe AP were admitted to the ICU immediately from the emergency department; of those, 2 (15%) died. Another 15 (45%) were transferred to the ICU from wards later during their hospitalization, of which 4 (26%) died, while five patients fulfilling criteria for severe AP were not treated in the ICU. Median time for transfer to the ICU was 4 days (range 1-6) from admission.

### Example 10

### Free Serum DNA Levels in Patients and Controls

When all AP patients were analyzed as a group, levels of free serum DNA were highly variable on all days of measurement, and were higher than those in the control group [median 0.079 ng/μl (0.010-0.231 ng/μl)]. This difference was not statistically significant, except on Day 4 [median 0.163 ng/μl (0.009-0.529 ng/μl); P=0.029].

There were no significant differences in serum DNA concentrations according to patients' age or sex, or between patients with alcoholic or biliary pancreatitis.

In patients who had mild disease, concentrations of serum DNA were not significantly different from the DNA concentrations in the control group. Patients who developed severe disease had significantly higher DNA concentrations than the control group on Day 1 (P<0.001) and Day 4 (P=0.008).

### Example 11

### Free Serum DNA Levels and Disease Severity

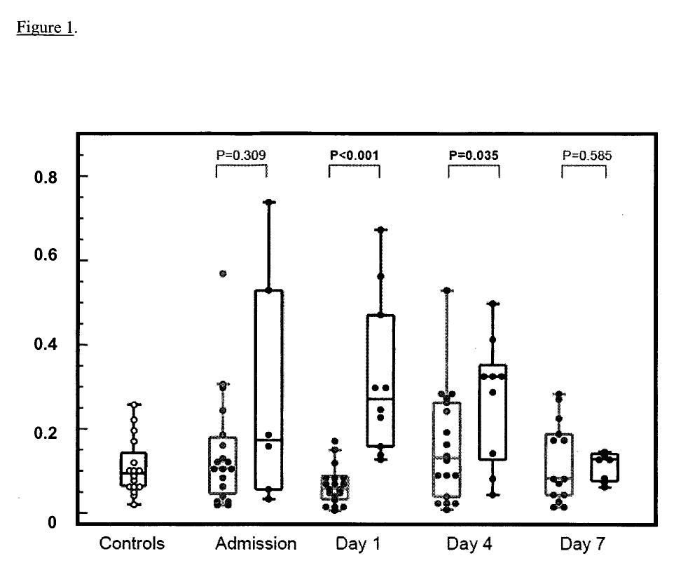

When levels of free serum DNA in patients who had mild AP were compared with patients who developed severe disease, the inventors found that the patients who would develop severe disease had higher DNA concentrations on all studied days as shown in FIG. 1 herein. The difference was most prominent on Day 1: patients with mild disease had median serum DNA level 0.059 ng/μl (0.007-0.170 ng/μl), while those with severe AP had median serum DNA level 0.27 ng/μl (0.128-0.672 ng/μl). This difference was statistically significant (P<0.001). Cell-free serum DNA level on Day 1 was thus tested as a potential predictor of disease severity:

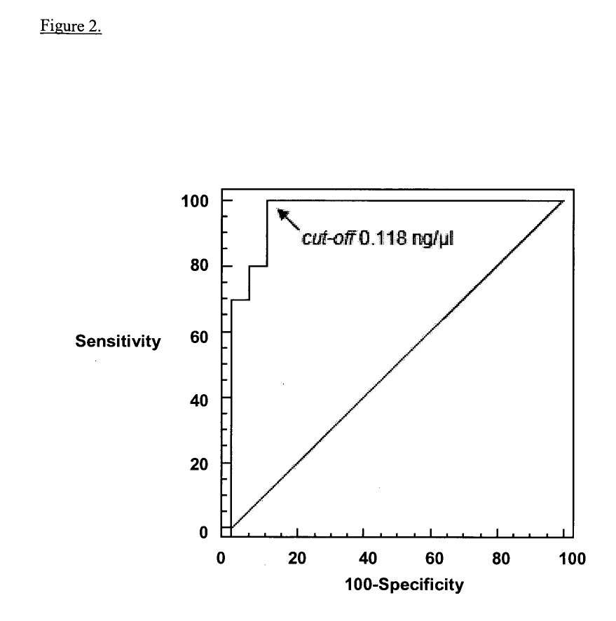

ROC curve (FIG. 2) showed very good characteristics, better than the predictors commonly used in use in clinical practice (Table 3). The cut-off value of 0.118 ng/μl showed best relationship between sensitivity (100%) and specificity (89.5%).

There was a correlation between serum DNA levels on the first day and highest APACHE II score of surviving patients (r=0.4674; P=0.0106), and also between serum DNA levels on the first day and length of hospitalization (r=0.4743; P=0.0093). There was no significant correlation with CRP levels (r=0.2421; P=0.2057).

### Example 12

### Free Plasma and Serum DNA Levels and Disease Severity

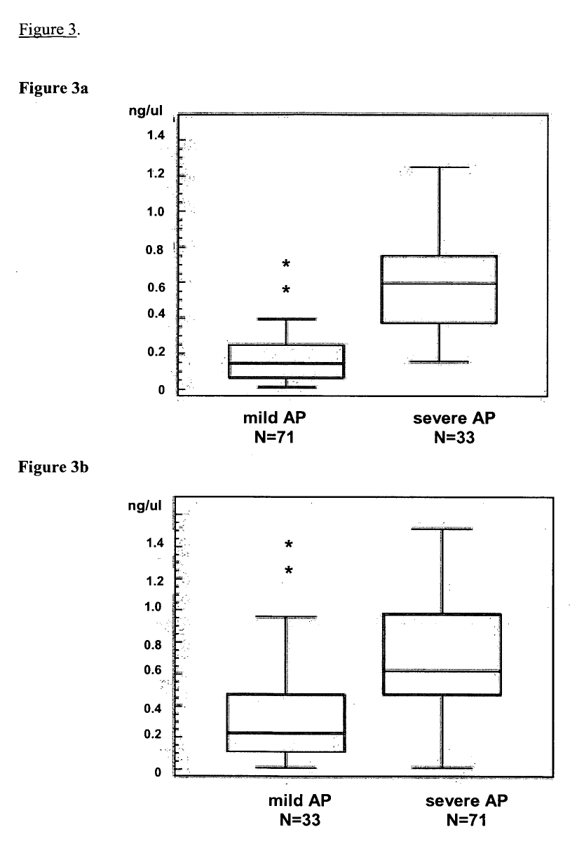

On the first day following admission, patients who had mild acute pancreatitis had significantly lower levels of free DNA compared to patients who would develop severe disease, in both plasma (FIG. 3a) and serum (FIG. 3b) samples. Median plasma free DNA level measured in patients with mild acute pancreatitis was 0.144 (0.012-0.66) ng/μl, while for severe patients it was 0.593 (0.157-1.250) ng/μl (P<0.001). For serum samples this difference was smaller: median free DNA level for patients with mild disease was 0.230 (0.015-0.230) ng/μl and for severe disease 0.623 (0.012-1.510) ng/μl (P<0.001).

Serum free DNA levels were in positive correlation with the highest CRP (r=0.266, P=0.007), highest APACHE II score (r=0.362, P=0.002) and total hospital LOS (r=0.321, P=0.001).

Plasma free DNA levels were also in positive correlation with the highest CRP (0.479, P<0.001), highest APACHE II score (r=0.559, P<0.001) and with total hospital LOS (r=0.465; P<0.001).

### Example 13

### Pancreatic Necrosis and Free DNA Levels

For the serum DNA study, abdominal CT was performed in 17 patients: 9 patients with severe disease and 8 patients with mild disease. There was a significant correlation between Balthazar score and free serum DNA concentrations (r=0.594, P=0.012).

For the comparative serum-plasma study, abdominal CT was performed in all 33 patients with severe disease and in 30 patients with mild disease. There was a significant correlation between Balthasar's score and both plasma (r=0.545, P<0.001) and serum (r=466, P<0.001) free DNA levels.

### Example 14

### Prediction of Severe Acute Pancreatitis

Table 3—Prediction of severe acute pancreatitis using Ranson's score (≧3), highest APACHE II score in the first 48 hours (≧5) CRP on the first two days and free serum DNA (≧0.118) on day one of hospitalization.

TABLE 3

| | Ranson's | APACHE II | | Free serum |
|---|---|---|---|---|
| | score | score | CRP | DNA |
| | (≧3) | (≧5) | (≧97) | (≧0.118 ng/μl) |
| Area under | 0.89 | 0.81 | 0.72 | 0.97 |
| ROC curve | (0.72-0.97) | (0.62-0.93) | (0.52-0.87) | (0.83-0.99) |
| (95% CI) | | | | |
| Sensitivity | 77.8% | 77.8% | 66.7% | 100% |
| (95% CI) | (40.1-96.5) | (40.1-96.5) | (30.1-92.1) | (69.0-100) |
| Specificity | 81.0% | 76.2% | 65.0% | 89.5% |
| (95% CI) | (58.1-94.4) | (52.8-91.7) | (40.8-84.5) | (66.8-96.4) |
| PPV | 63.6% | 58.3% | 46.2% | 83.3% |
| NPV | 89.5% | 88.9% | 81.2% | 100% |
| Positive | 4.08 | 3.27 | 1.9 | 9.50 |
| likelihood | | | | |
| ratio | | | | |
| Negative | 0.27 | 0.29 | 0.51 | 0 |
| likelihood | | | | |
| ratio | | | | |

APACHE II—Acute Physiology and Chronic Health Evaluation (APACHE) II score; CRP—C-reactive protein; CI—confidence interval; ROC—receiver operating characteristic; PPV—positive predictive value; NPV—negative predictive value

### Example 15

### Table 4—Receiver Operating Curve Analyses

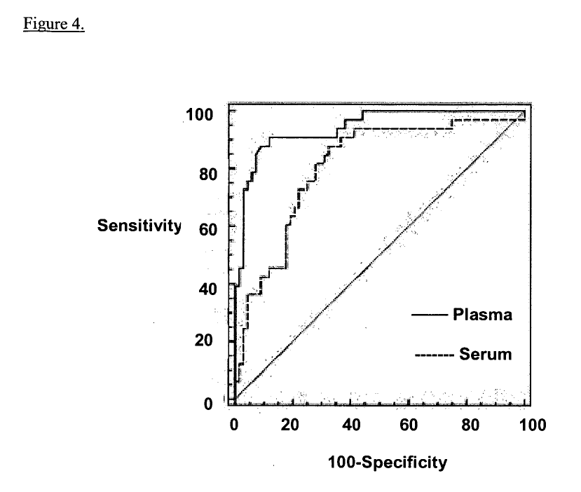

Receiver operating characteristic (ROC) curves were created from results for both plasma and serum free DNA levels (FIG. 4). Results showed that plasma free DNA levels differentiate between patients with mild and severe acute pancreatitis with great sensitivity and specificity (90.9%, 88.7% respectively, for cut-off value of >0.304 ng/μl) (Table 4). Although results for free serum DNA levels were a bit poorer (sensitivity 87.9%, specificity 67.6% for cut-off>0.363 ng/μl), both plasma and serum free DNA showed better sensitivity and specificity than CRP or two scoring systems (Ranson's and APACHEII score) that are in practice in most usual clinical predictors for severity of acute pancreatitis.

TABLE 4
Receiver operating characteristics of plasma and
serum free DNA as predictors of severity of acute
pancreatitis on the first day after admission

| | Free plasma DNA | Free serum DNA |
|---|---|---|
| | (≧0.304 ng/μl) | (≧0.363 ng/μl) |
| Area under ROC curve | 0.942 | 0.809 |
| (95% CI) | (0.878-0.978) | (0.718-0.880) |
| Sensitivity (95% CI) | 90.9% | 87.9% |
| | (75.6-98.0) | (71.8-96.5) |
| Specificity (95% CI) | 88.7% | 67.6% |
| | (76.7-95) | (55.2-78.5) |
| PPV | 78.9 | 40.4 |
| NPV | 95.5 | 95.7 |
| Positive likelihood ratio | 8.07 | 2.72 |
| Negative likelihood ratio | 0.10 | 0.18 |

CI—confidence interval; ROC—receiver operating characteristic; PPV—positive predictive value; NPV—negative predictive value

### Example 16

### Overall

The inventors tested both plasma and serum free DNA levels as prognostic markers for severity of acute pancreatitis. Results show that cell-free DNA measured in both plasma and serum of patients with acute pancreatitis on the first day after hospital admission is significantly higher in patients who developed severe pancreatitis than in those with mild disease.

The inventors included all patients admitted with primary admission diagnosis of acute pancreatitis (regardless of duration of symptoms and other acute or chronic conditions). Patients with terminal illness or in whom pancreatitis was a secondary diagnosis were still excluded since in them the course of disease and hospitalization depended on the primary condition. The included population was shown to be of a great value since there was no statistically significant difference between patients with mild or severe disease in their sex or age distribution, or aetiology of pancreatitis. The occurrence of severe cases in the population used was 32% which is even greater than usual (around 20%), which improved the statistical analyses.

Since previous work on free DNA suggests that plasma samples give more accurate results of free DNA concentrations (due to sample handling method), the inventors included plasmas as additional samples and compared levels of free DNA concentrations to those of sera.

The source of free circulating DNA in acute conditions previously studied is not clearly defined, although cell-death (apoptotic or necrotic) is probably the primary source (Gormally et al. Mutation Research 2007; 635:105-17; Tong et al. Clinica Chimica Acta; International J of Clin Chem 2006; 363:187-96; Giacona et al. Pancreas 1998; 17:89-97). Free DNA in acute pancreatitis is also supposed to originate from the necrosis of pancreatic tissues and therefore patients with mild disease who have limited or no necrosis are expected to have lower DNA concentrations than patients with severe course in whom pancreatic necrosis of greater extent causes significant influx of free DNA in the circulation. Positive correlation of both serum and plasma DNA levels with the extent of pancreatic necrosis (Balthasar's score) were in concordance.

According to the results, both plasma and serum levels of free DNA predict severity of acute pancreatitis better than other common markers and scoring systems.

Another great value of this predictor is that it can predict disease severity on the first day following admission to a hospital; that is, it can be used for early detection, diagnosis and/or prognosis of AP, whether assessed at a hospital or another location. The practical value of this is apparent if one looks at the delay in recognizing patients with severe disease. For about 40% of the patients it was apparent at presentation that they had severe disease and they were immediately admitted to ICU, but even a higher number of patients who were eventually diagnosed with severe AP were admitted to wards and were transferred to ICU with a median delay of 4 days. A marker recognising severe AP on the first day after admission could have reduced this delay for a median three days. Early intensive treatment improves outcome in severe acute pancreatitis (Besselink et al. 2007; Curr Opin Crit Care 13:200-6), which is also apparent from the observation that delayed admission to ICU was associated with higher mortality.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method of determining the likelihood of acute pancreatitis (AP) development in an individual, comprising:
obtaining a sample from the individual;
assaying the sample to detect the presence or absence of a significantly high level of cell-free DNA in the sample;
determining a high risk of AP development in the individual based upon the presence of a significantly high level of cell-free DNA in the sample or diagnosing a low risk of AP development in the individual based upon the absence of a significantly high level of serum DNA in the sample.

2. The method of claim 1, wherein the sample is a serum sample.

3. The method of claim 1, wherein the sample is a plasma sample.

4. The method of claim 1, wherein the sample is a combination of a serum sample and a plasma sample.

5. The method of claim 1, wherein the significantly high level of cell-free DNA is a level above 0.050 ng/μl.

6. The method of claim 1, wherein the significantly high level of cell-free DNA is a level above 0.100 ng/μl.

7. The method of claim 1, wherein the significantly high level of cell-free DNA is a level above 0.150 ng/μl.

8. The method of claim 1, wherein the significantly high level of cell-free DNA is a level of at least 0.271 ng/μl.

9. The method of claim 1, wherein the significantly high level of cell-free DNA is a level of at least 0.304 ng/μl.

10. The method of claim 1, wherein the significantly high level of cell-free DNA is a level of at least 0.363 ng/μl.

11. The method of claim 1, wherein the AP is a severe form of AP.

12. The method of claim 1, further comprising detecting gallstones in the individual prior to obtaining the sample.

13. The method of claim 1, further comprising detecting one or more stimulants in the individual prior to obtaining the sample.

14. The method of claim 13, wherein the stimulants are selected from the group consisting of toxins, drugs, infections, trauma, vascular insults, anatomic abnormalities, metabolic derangements and combinations thereof.

15. The method of claim 1, further comprising detecting hypertriglyceridemia and/or hypercalcemia in the individual prior to obtaining the sample.

16. The method of claim 1, wherein determining a high risk of AP development in the individual further comprises determining a likelihood of developing a related complication selected from the group consisting of pancreatic necrosis, tissue abscess, glandular damage, and diabetes mellitus.

17. The method of claim 1, wherein the sample is obtained from the individual on the day that the individual is admitted to a hospital.

18. A method of determining the prognosis of a subject with pancreatitis, comprising:
obtaining a sample from the subject;
assaying the sample to detect the presence or absence of a significantly high level of cell-free DNA in the sample relative to either a healthy individual or an individual who has and maintains a mild form of acute pancreatitis; and
prognosing a form of pancreatitis that will progress to severe AP based upon the presence of the significantly high level of cell-free DNA in the subject relative to either the healthy individual or the individual who has and maintains a mild form of acute pancreatitis.

19. The method of claim 18, wherein the sample is a serum sample.

20. The method of claim 18, wherein the sample is a plasma sample.

21. The method of claim 18, wherein the sample is a combination of a serum sample and a plasma sample.

22. The method of claim 18, wherein the significantly high level of cell-free DNA is a level above 0.050 ng/μl.

23. The method of claim 18, wherein the significantly high level of cell-free DNA is a level above 0.100 ng/μl.

24. The method of claim 18, wherein the significantly high level of cell-free DNA is a level above 0.150 ng/μl.

25. The method of claim 18, wherein the significantly high level of cell-free DNA is a level of at least 0.271 ng/μl.

26. The method of claim 18, wherein the significantly high level of cell-free DNA is a level of at least 0.304 ng/μl.

27. The method of claim 18, wherein the significantly high level of cell-free DNA is a level of at least 0.363 ng/μl.

28. The method of claim 18, wherein the sample is obtained from the individual on the day that the individual is admitted to a hospital.

29. A method of treating acute pancreatitis in a subject, comprising:
obtaining a sample from the subject;
assaying the sample to detect the presence or absence of a significantly high level of cell-free DNA in the sample;
determining a high risk of AP development in the individual based upon the presence of a significantly high level of cell-free DNA in the sample or diagnosing a low risk of AP development in the individual based upon the absence of a significantly high level of serum DNA in the sample; and
treating the subject to either prevent or lessen the likelihood of developing or mitigate the effects of AP.

30. The method of claim 29, wherein the sample is a serum sample.

31. The method of claim 29, wherein the sample is a plasma sample.

32. The method of claim 18, wherein the sample is a combination of a serum sample and a plasma sample.

33. The method of claim 29, wherein the significantly high level of cell-free DNA is a level above 0.050 ng/μl.

34. The method of claim 29, wherein the significantly high level of cell-free DNA is a level above 0.100 ng/μl.

35. The method of claim 29, wherein the significantly high level of cell-free DNA is a level above 0.150 ng/μl.

36. The method of claim 29, wherein the significantly high level of cell-free DNA is a level of at least 0.271 ng/μl.

37. The method of claim 29, wherein the significantly high level of cell-free DNA is a level of at least 0.304 ng/μl.

38. The method of claim 29, wherein the significantly high level of cell-free DNA is a level of at least 0.363 ng/μl.

39. The method of claim 29, wherein the sample is obtained from the individual on the day that the individual is admitted to a hospital.

40. A kit for determining the likelihood of acute pancreatitis (AP) development in an individual, comprising:
one or more components to obtain a blood sample from the individual;
one or more components to isolate plasma or serum or both from the blood sample;
one or more components to determine a level of plasma DNA or serum DNA; and
instructions with tangible expression describing a technique, comprising
obtaining the blood sample from the individual,
isolating plasma or serum from the blood sample,
determining a significantly high level of plasma DNA or serum DNA in the blood sample, and
determining a high risk of AP development in the individual based upon the presence of a significantly high level of plasma DNA or serum DNA in the blood sample.

41. A kit for treating acute pancreatitis (AP) in an individual, comprising:
one or more components to obtain a blood sample from the individual;
one or more components to isolate plasma or serum or both from the blood sample;
one or more components to determine a level of plasma DNA or serum DNA;
instructions with tangible expression describing a technique, comprising
obtaining the blood sample from the individual,
isolating plasma or serum from the blood sample,
determining a significantly high level of plasma DNA or serum DNA in the blood sample, and
determining a high risk of AP development in the individual based upon the presence of a significantly high level of plasma DNA or serum DNA in the blood sample.

42. A method of assessing the severity of episodes of AP in patients with chronic pancreatitis, who have recurrent episodes of AP, comprising:
obtaining a sample from the subject;
assaying the sample to detect the presence or absence of a significantly high level of cell-free DNA in the sample relative to either a healthy individual or an individual who has and maintains a mild form of acute pancreatitis; and
determining the severity of episodes of AP based upon the presence of the significantly high level of cell-free DNA in the subject relative to either the healthy individual or the individual who has chronic pancreatitis.

43. The method of claim 42, wherein the sample is a serum sample.

44. The method of claim 42, wherein the sample is a plasma sample.

45. The method of claim 42, wherein the sample is a combination of a serum sample and a plasma sample.

46. The method of claim 42, wherein the significantly high level of cell-free DNA is a level above 0.050 ng/μl.

47. The method of claim 42, wherein the significantly high level of cell-free DNA is a level above 0.100 ng/μl.

48. The method of claim 42, wherein the significantly high level of cell-free DNA is a level above 0.150 ng/μl.

49. The method of claim 42, wherein the significantly high level of cell-free DNA is a level of at least 0.271 ng/μl.

50. The method of claim 42, wherein the significantly high level of cell-free DNA is a level of at least 0.304 ng/μl.

51. The method of claim 42, wherein the significantly high level of cell-free DNA is a level of at least 0.363 ng/μl.

52. The method of claim 42, wherein the sample is obtained from the individual on the day that the individual is admitted to a hospital.
